SEQUENCE LISTING (6 sequences)

SEQ ID NO: 1 - DNA, 21 bp, Artificial Sequence
  Inventor Kanbara, Hideki Inventor Kishimoto, Akihiko Inventor Tomoharu, Kajiyama Inventor Zhou, Guohua
  Description of Artificial Sequence primer
aaggcactcttgcctacgcca

SEQ ID NO: 2 - DNA, 71 bp, Artificial Sequence
  Description of Artificial Sequence template DNA
gactgaatataaacttgtggtagttggagctgttggcgtaggcaagagtgccttgacgatacagctaattc

SEQ ID NO: 3 - DNA, 33 bp, Artificial Sequence
  Description of Artificial Sequence sequence to be analyzed
acagctccaactaccacaagtttatattcagtc

SEQ ID NO: 4 - DNA, 22 bp, Artificial Sequence
  Description of Artificial Sequence primer
gaacagctttgaggtgcgtgtt

SEQ ID NO: 5 - DNA, 190 bp, Artificial Sequence
  Description of Artificial Sequence template DNA
ctttcttgcggagattctcttcctctgtgcgccggtctctcccaggacaggcactaacacgcacctcaaagctgttccgtcccagtagattaccaaccattagatgaccctgccttgtcgaaactccacgcacaatcacggacaggaccctctctggccgcgtgtctccttctcttagaggcgttctttc

SEQ ID NO: 6 - DNA, 55 bp, Artificial Sequence
  Description of Artificial Sequence sequence to be analyzed
agtgcctgtcctgggagagaccggcgcacagaggaagagaatctccgcaagaaag

(19) United States
(12) Patent Application Publication
(10) Pub. No.: US 2007/0054283 A1
(43) Pub. Date: Mar. 8, 2007

(54) DNA sequencing method using step by step reaction

(75) Inventors: Akihiko Kishimoto, Tachikawa (JP); Hideki Kambara, Hachioji (JP); Tomoharu Kajiyama, Higashiyamato (JP); Guohua Zhou, Kodaira (JP)

Correspondence Address: Stanley P. Fisher;Reed Smith LLP, Suite 1400, 3110 Fairview Park Drive, Falls Church, VA, 22042-4503, US

(73) Assignee: Hitachi, Ltd.

(21) Appl. No.: 11/356,187

(22) Filed: Feb. 17, 2006

(30) Foreign Application Priority Data
Sep. 6, 2005 (JP) 2005-257926

(51) Int. Cl. C12Q 1/68

(52) U.S. Cl. 435/6

(57) ABSTRACT

The present invention provides an inexpensive DNA sequencing method with high-sensitivity. The method of the present invention comprising the steps of, adding an given amount of dATP for step by step complementary strand synthesis and subtracting the background luminescence intensity caused by dATP from the measured luminescence intensity to obtain the luminescence intensity involved in complementary strand synthesis.

### CLAIM OF PRIORITY

The present application claims priority from Japanese application JP 2005-257926 filed on Sep. 6, 2005, the content of which is hereby incorporated by reference into this application.

### FIELD OF THE INVENTION

The present invention relates to a DNA sequencing method using chemical luminescence. More specifically, the present invention relates to a DNA sequencing method using chemical luminescence, which enables inexpensive and high-sensitivity DNA sequencing with minimized effect of background luminescence caused by dATP.

### BACKGROUND OF THE INVENTION

A method using gel electrophoresis and DNA detection using fluorescence is commonly adopted in DNA sequencing. This method involves several steps. First of all, DNA fragments, of which sequences are to be analyzed, are amplified. Second, DNA fragments of different lengths starting at their 5′ terminals are prepared and fluorescence labels of different wavelengths are added to their 3′ terminals depending on the kinds of bases. Third, any differences in length are identified among fluorescence-labeled fragments in increments of one base, while the types of the bases at the 3′ terminals are determined based on fluorescent colors emitted by individual fragment groups. The DNA fragments sequentially pass through a fluorescent detector beginning from a shortest one and therefore, the measurement of fluorescent colors enables the types of the bases at the terminals to be sequentially determined beginning from the shortest one, successfully achieving sequencing. A fluorescent DNA sequencer based on this principle has become widely used and played an important role in the Human Genome Project (Gendai Kagaku, July 2004, vol.400, p66-69).

Since it was declared in 2003 that sequence analysis had been finished in the Human Genome Project, sequence information has been used in the medical field, as well as various kinds of industries. Recently, we need not analyze a long DNA sequence of total-length of DNA, but it is enough to determine a limited target sequence region of DNA in many cases. In this context, the need for a convenient method and an apparatus suitable for such an analysis of the short DNA sequence has arisen.

To satisfy this need, sequencing methods based on step by step complementary strand synthesis represented by pyrosequencing have been developed. These methods comprises the steps of: hybridizing a primer with a target DNA strand and sequentially adding four types of nucleic acid substrates (dATP, dCTP, dGTP, and dTTP) for complementary strand synthesis in the reaction solution one by one to induce complementary strand synthesis. Once complementary strand synthesis has started, pyrophosphoric acid (PPi) is released as a reaction product. In pyrosequencing, PPi reacts with APS (adenosine 5′-phosphosulfate) in the presence of ATP sulfurylase, resulting in the production of ATP, which in turn, reacts with luciferin in the presence of luciferase, emitting light (hereinafter, simply referred to as luminescence involved in complementary strand synthesis). Then, the detection of resultant luminescence provides information indicating that the added nucleic acid substrates for complementary strand synthesis have been incorporated into the DNA strand, and thereby enabling sequencing of the target DNA strand (Electrophoresis, 22, pp. 3497-3504 (2001). The residual component of nucleic acid for the complementary strand synthesis, which has not been used in the synthesis reaction, is rapidly degraded in the presence of an enzyme (e.g. apyrase) and thereby avoiding its effect on the subsequent steps.

Initially, such a pyrosequencing method was reported that the complementary strand synthesis and chemical luminescence reactions were made in different reaction vessels (U.S. Pat. No. 4,863,849). In other words, a reaction solution containing pyrophosphoric acid produced during complementary strand synthesis and excessive amounts of nucleic acid substrate was moved from the reaction vessel, in which complementary strand synthesis was carried out, to another vessel to induce a luminescence -reaction. In addition, another method was reported, which involved steps of passing the reaction solution through a region, where an degrading enzyme of the excessive amounts of nucleic acid substrate has been immobilized, in mid course of the degrading and transforming pyrophosphoric acid into ATP followed by introducing the resultant ATP into the chemical luminescence reaction vessel (U.S. Pat. No.4,971,903). This method, however, further requires a step of washing the vessel followed by replacing the old solution with new one every time of adding nucleic acid, the substrate for complementary strand synthesis, making the process more complicated.

To solve this problem, a convenient method was proposed and has been commonly used, which has integrated together four reactions, namely complementary strand synthesis, the degradation of excessive amounts of nucleic acid substrate, ATP production, and luminescence (U.S. Pat. No.6,258,568). dATP to be used in complementary strand synthesis, however, has a structure similar to that of ATP and therefore, it acts as the substrate for luciferase reaction, causing chemical luminescence. The chemical luminescence has intensity far lower than that of ATP but can not be neglected in DNA sequencing because it always induces background luminescence (hereinafter, simply referred to as background luminescence caused by dATP) every time dATP is injected. To solve this problem, recently, dATPαS, which does not act as the substrate for the chemical luminescence reaction and can be used in DNA complementary strand synthesis, has been used as the substrate for complementary strand synthesis instead of dATP (U.S. Pat. No. 6,210,891 and JP Patent No. 3510272). On the other hand, this method has a disadvantage in that the reagent is more expensive than dATP and lower in reactivity with the template DNA with a higher probability of dATPαS being left un-reacted than those of other types of substrates for complementary strand synthesis. Accordingly, the need for a more inexpensive and higher-accuracy method for sequencing has arisen.

On the other hand, an increased concentration of luciferase augments the amount of luminescence and thereby, it is useful in improving the sensitivity to detect a trace amount of ATP. In contrary, in pyrosequencing, the increased concentration enhances background luminescence caused by APS, as well as impurities in the reagent and thereby it is not effective. A decreased amount of APS attenuates the signal intensity to be measured and therefore, a limit to signal detection can not be lowered as long as APS is used, meaning that the APS concentration governs the limit to signal detection.

To solve the problems mentioned above, further another method using pyruvate phosphate dikinase (PPDK) is known, by which ATP is produced form PPi instead of APS. For example, such a method has been reported that PPi produced in polymerase chain reaction (PCR) is transformed into ATP under the presence of AMP and PPDK, and luminescence is detected through a luciferin-luciferase reaction (Analytical Biochemistry 268, pp. 94-101 (1999)).

### SUMMARY OF THE INVENTION

An object of the present invention is to provide a DNA sequencing method using inexpensive and high-sensitivity step by step complementary strand synthesis instead of an expensive and low-reactivity reagent, dATPαS.

To attain the object mentioned above, according to the present invention, the amount of background luminescence caused by dATP is decreased and background luminescence caused by dATP is subtracted from measured luminescence to obtain the luminescence intensity involved in complementary strand synthesis. In particular, when both the peaks of background luminescence caused by dATP and of measured luminescence appear almost at the same time, the peak luminescence intensity caused by dATP is subtracted from the peak intensity of measured luminescence to obtain the luminescence intensity involved in complementary strand synthesis. In addition, if any time lag lies between luminescence caused by dATP and measured luminescence, the time lag is used to obtain the luminescence intensity involved in complementary strand synthesis. dATP is injected into the reaction vessel at least two times, then the luminescence intensities caused by dATP under two conditions, namely with or without complementary strand synthesis, are monitored, and finally only the signal intensity involved in complementary strand synthesis is extracted to achieve accurate DNA sequencing.

The present invention enables DNA sequencing using step by step complementary strand synthesis without an expensive reagent.

The present invention may be used in gene diagnosis and gene displacement detection by means of DNA sequencing and the identification of the kinds of nucleic acid bases.

### BRIEF DESCRIPTION OF THE DRAWINGS

FIG. 1 is a principle of the step by step sequencing.

FIG. 2 shows an enzymatic reaction in the step by step sequencing.

FIG. 3 shows luciferase luminescence intensity caused by ATP and dATP.

FIGS. 4A, 4B, 4C, 4D, 4E, and 4F show the luminescence intensity profile dependent on the amount of template DNA with or without complementary strand synthesis. The ratios of dATP to the template DNA are as follows: 100 times in FIG. 4A, 50 times FIG. 4B, 20 times in FIG. 4C, 10 times in FIG. 4D, 5 times in FIG. 4E, and 2.5 times in FIG. 4F.

FIG. 5 shows peak values of the luminescence intensity dependent on the amount of template DNA due to complementary strand synthesis and the background luminescence intensity.

FIGS. 6A and 6B show a luminescence intensity pattern (pyrogram) obtained by single injection; FIG. 6A shows the result of measurement, and FIG. 6B shows the signal intensity profile after subtraction.

FIGS. 7A, 7B, 7C, 7D, and 7E show the luminescence intensity profile dependent on the temperature with or without complementary strand synthesis: the temperatures are 15° C., 20° C., 25° C., 30° C., 35° C., and 43° C., respectively.

FIG. 8 shows arriving time to the peak dependent on the temperature with and without the template DNA.

FIGS. 9A, 9B, and 9C show the determining method of the cut off time and the luminescence intensity profile after correction: in FIG. 9A, the luminescence intensity profile and the cut off time are shown and in FIG. 9B, the luminescence intensity profile after correction with the template DNA is shown, and in FIG. 9C, the luminescence intensity profile after correction without the template DNA.

FIGS. 10A and 10B show the luminescence intensity pattern (pyrogram) obtained after twice injections; in FIG. 10A, the result of measurement is shown and in FIG. 10B, the signal intensity profile after subtraction.

FIG. 11 shows the relationship between the luminescence intensity involved in complementary strand synthesis and the number of injections when the number of base duplications.

FIGS. 12A and 12B show the method for obtaining the luminescence intensity profile involved in complementary strand synthesis; in FIG. 12A, the measured luminescence intensity profile and the background luminescence intensity profile are shown and in FIG. 12B, the luminescence intensity profile involved in complementary strand synthesis is shown.

### DETAILED DESCRIPTION OF THE PREFERRED EMBODIMENTS

According to the present invention, an amount, which does not exceed 50 times the amount of a template DNA, of dATP is added in the reaction vessel containing the template DNA for step by step synthesis and the luminescence intensity caused by dATP is subtracted from the measured luminescence intensity to obtain the luminescence intensity involved in complementary strand synthesis for sequencing of the template nucleic acid.

The amount of dATP to be injected into the reaction vessel does not preferably exceed 20 times the amount of the template DNA and is more preferably 2.5 to 20 times.

According to a first embodiment of the present invention, a background luminescence intensity profile caused by dATP is subtracted from a measured luminescence intensity profile to obtain a luminescence intensity profile involved in complementary strand synthesis.

According to a second embodiment of the present invention, the peak luminescence intensity caused by dATP is subtracted from the peak intensity of measured luminescence to obtain the peak luminescence intensity involved in complementary strand synthesis.

According to a third embodiment of the present invention, an area of the luminescence intensity profile caused by dATP is subtracted from an area of the measured luminescence intensity profile to obtain a luminescence intensity profile involved in complementary strand synthesis.

According to a fourth embodiment of the present invention, when a time lag lies between luminescence caused by dATP and measured luminescence, the time lag is used for removing background caused by dATP to obtain luminescence involved in complementary strand synthesis.

Namely, the measured luminescence intensity profile is compared with the background luminescence intensity profile caused by dATP to detect a point where the peaks of both luminescences can be suitably discriminated. It is assumed that the time passed to reach the point starting at the time when dATP is injected is a cut off time, the luminescence intensity profile up to the cut off time is 0 (zero), and the luminescence intensity profile since the cut off time is the luminescence signal intensity involved in complementary strand synthesis. Namely, in measuring the intensity of chemical luminescence involved in complementary strand synthesis, the signal intensity generating in the time period from the point where dATP is injected to the cut off time are removed to obtain the signal intensity involved in complementary strand synthesis.

According to a fifth embodiment of the present invention, a variation in intensity of chemical luminescence, which is achieved by repeating a step of injecting dATP into the reaction vessel several times, is used to obtain the signal intensity involved in complementary strand synthesis. Namely, dATP is injected into the reaction vessel at least two times to confirm background luminescence caused by dATP every time dATP is injected and the background luminescence intensity caused by dATP is subtracted from the measured luminescence intensity to obtain the luminescence intensity involved in complementary strand synthesis.

In the method of the present invention, it is preferable that excessive amounts of dNTP is removed after complementary strand synthesis to converge chemical luminescence in the given time period. This type of removal of the nucleic acid substrate may be done in the presence of the enzyme (apyrase) mixed with the reaction solution in the reaction vessel or may be done by immobilizing the template DNA and the nucleic acid synthase and then replacing the reaction solution with new one in the reaction vessel.

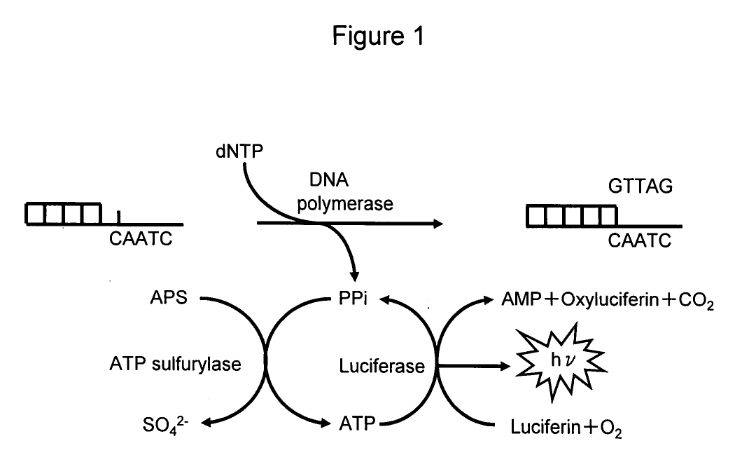

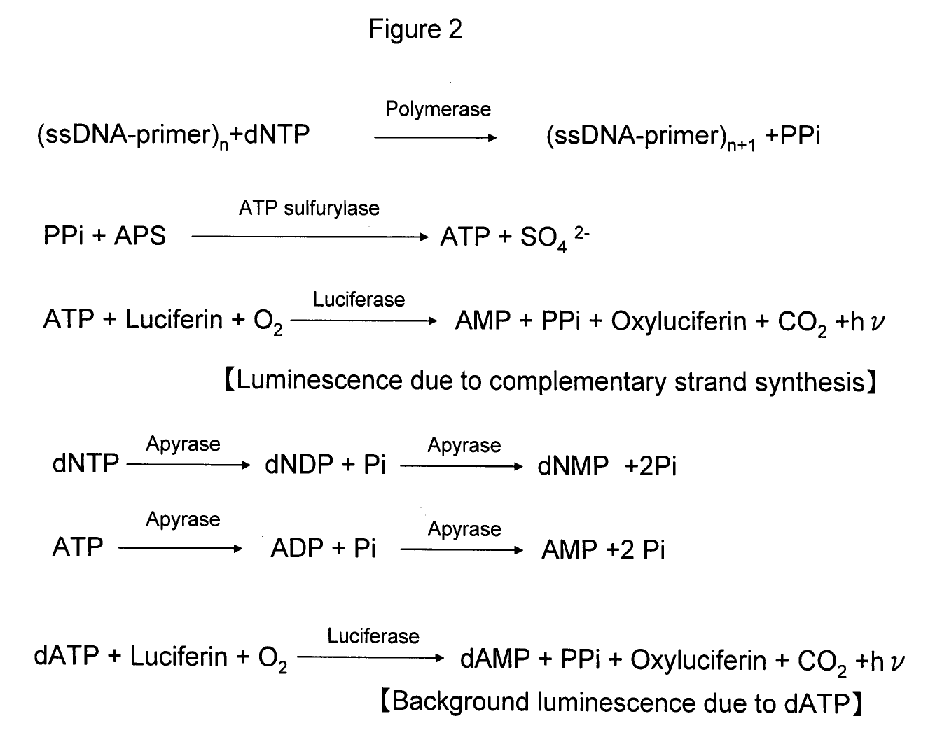

The principle of the pyrosequencing method is shown in FIG. 1 and the reaction of the enzyme used herein is shown in FIG. 2, respectively. In the pyrosequencing method, PPi (pyrophosphoric acid, inorganic pyrophosphate) produced through the DNA base elongation reaction is transformed into ATP and the resultant ATP is luminously reacted with luciferin, emitting light to be detected. The basic enzymatic reaction in the pyrosequencing involves four reactions in the presence of enzymes; 1) step by step DNA complementary strand synthesis, 2) transformation of the product PPi into ATP, 3) reaction of ATP with luciferin to induce luminescence, and 4)degradation and removal of excessive amounts of substrate (nucleic acid) for complementary strand synthesis, which is usually added to the former three reactions.

The reactions in the pyrosequencing are briefly described below by reference to FIG. 1. To synthesize the target DNA strand using a primer assuming that the complementary strand DNA is the template, four kinds of substrates (A, C, G, T) (generically referred to as dNTP (Deoxynucleotide triphosphate)) for complementary strand synthesis are sequentially injected into the reaction vessel. In FIG. 1, if the injected substrate is dGTP, the substrate dGTP is used in an elongation reaction using DNA as the template, elongating the primer because of its. complementarity with an elongation site C of the template DNA. PPi produced by this reaction reacts with Adenosine 5′ phosphosulfate (APS) in the presence of an enzyme ATP sulfurylase to produce ATP. The resultant ATP reacts with luciferin in the presence of the enzyme luciferase, inducing the luminescence reaction.

In this reaction, PPi is reproduced at the same time luminescence is excited and therefore, individual cycle reactions in the presence of two enzymes, namely ATP sulfurylase and luciferase, respectively are induced, luminescence being retained. The target DNA strand is reproduced by complementary strand synthesis and thereby, the kind of the base, which induces complementary strand synthesis when injected, is monitored by means of the presence or absence of luminescence to determine the base sequences. For example, if light is emitted when dGTP is injected, the DNA sequence at the target site is G.

It is required that the nucleic acid substrate injected in the reaction vessel have been removed from there before the initiation of a step of injecting the next nucleic acid substrate. To do so, the enzyme or DNA may be immobilized on bead surfaces or the like and be left in the reaction vessel followed by replacing the reaction solution with new one. Alternatively, a method, by which the enzyme apyrase is used to degrade phosphate groups of ATP and dNTP for inactivation, is commonly used as a convenient one. In the examples described below, the nucleic acid substrate was degraded with the aid of apyrase.

Four kinds of substrates are sequentially injected into the reaction vessel. If the injected substrate is complementary with the elongation site of the template DNA, complementary strand synthesis proceeds and luminescence involved in complementary strand synthesis is detected, while if the injected substrate is not complementary, no luminescence is detected. The substrate for complementary strand synthesis substrate, which is left un-reacted, and ATP are degraded in the presence of apyrase and do not involve in subsequent reactions. This step is repeated until the DNA base sequences are determined.

The nucleic acid substrates used in a conventional pyrosequencing method are dATPαS, dCTP, dGTP, and dTTP. In a fluorescent DNA sequencing method, which is commonly used, dATP is used in DNA complementary strand synthesis, while in the pyrosequencing method, dATPαS has been generally used. It is because unlike dNTP, dATP has a structure similar to that of ATP and therefore, reacts with luciferin in the presence of luciferase with reactivity lower than that of ATP to produce background luminescence caused by dATP. In usual complementary strand synthesis, an amount, which is more than several tens times the amount of the template DNA, of substrate for complementary strand synthesis is injected into the reaction vessel. For example, in JP Patent Publication (Kohyo) No. 2001-506864, an amount, which is 80 times the amount of template DNA, was added into the reaction vessel.

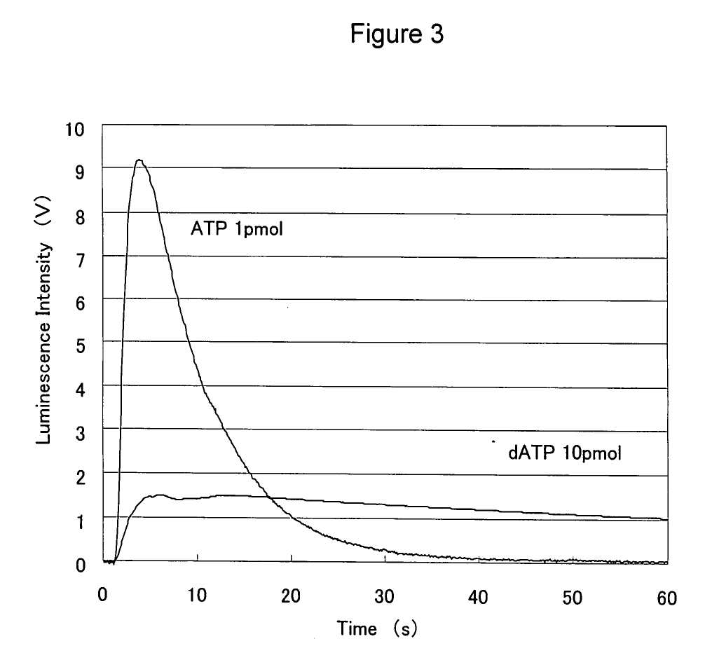

Considering that a plurality of bases of the same kind are trapped at the same time, it is naturally enough to induce luminescence that the concentration of dNTP is several times that of the template DNA. In this example, the progress of the complementary strand synthesis reaction was discussed for each of various concentrations and it was demonstrated that it was enough to induce luminescence that the concentration of dNTP was ten times or less that the template DNA. FIG. 3 shows the intensity of the luciferase luminescence reaction when 1 pmol of ATP was injected and when 10 pmol of dATP was injected for comparison. As shown in the figure, dATP may also act as a substrate of the luciferase luminescence reaction and the background luminescence intensity caused by dATP is ⅙ the background luminescence intensity caused by ATP when the amount of dATP is ten times the amount of ATP. It should be noted that under an actual pyrosequencing condition, a degradation reaction is induced in the presence of apyrase and therefore, a difference in peak signal intensities becomes small. As mentioned above, in pyrosequencing, the amount of ATP produced by complementary strand synthesis is at least approximately the same as that of the target DNA, while under a conventional pyrosequencing condition, an amount, which is several tens times that of current pyrosequencing, of dNTP was added. When this amount of dNTP is added, the background luminescence intensity caused by dATP is high and luminescence involved in DNA complementary strand synthesis, as well as pyrophosphoric acid production and ATP production involved in the synthesis is offset, raising a severe problem in sequencing. To solve this problem, in the conventional method, dAYPαS with low chemical luminescence reactivity has been used to induce complementary strand synthesis.

### EXAMPLES

Hereinafter, the present invention is described in detail by reference to examples but the present invention is not limited only to these examples.

### Example 1

In this Example 1, a method, by which the background luminescence intensity profile caused by dATP is subtracted from the measured luminescence intensity profile to obtain the luminescence intensity profile involved in complementary strand synthesis, is described.

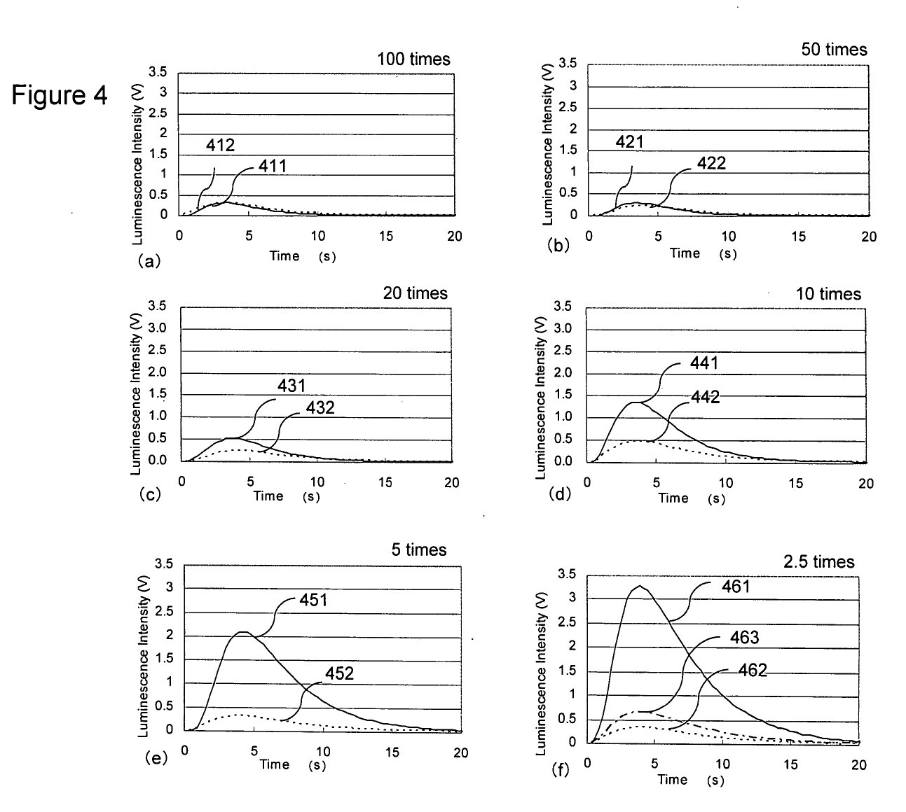

FIG. 4 shows a variation in the luminescence signal intensity depending on the amount of the template DNA with or without complementary strand synthesis. An amount of dATP for each injection was 25 pmol. 25 mpol of dATP was consecutively injected into the reaction vessel four times. The injected dATP was degraded in the presence of apyrase every time being injected after the complementary strand synthesis. A p53 variant was used for the template DNA. The amount of the template DNA injected was varied: 0.25 pmol, 0.5 pmol, 1.25 pmol, 2.5 pmol, 5 pmol, and 10 pmol in that order. For all these amounts of the template DNA, experiments were performed at room temperature, 25° C. The ratios of dATP to the template DNA for each reaction were; 100 times, 50 times, 20 times, 10 times, 5 times, and 2.5 times, being expressed in FIGS. 4A, 4B, 4C, 4D, and 4F, respectively.

Giving an example of a case (d), in which the amount of the template DNA was 2.5 pmol and the ratio of dATP to the template DNA was 10 times, the experiment result is explained as below. 25 pmol of dATP was consecutively injected four times. A first luminescence intensity profile is indicated by a solid line 441. After second to fourth dATP injections, no variation was observed in the luminescence intensity profile. This is expressed by a dotted line 442. It may be suggested that no variation was observed in the luminescence intensity profile after the second to fourth dATP injections because complementary strand synthesis of the template DNA sufficiently proceeded after initial dATP injection and no template DNA remained un-reacted after the second and consecutive dATP injections, only background luminescence due to dATP was observed.

Solid lines 411, 421, 431, 441, 451, and 461 indicate both of luminescence involved in DNA complementary strand synthesis and luminescence caused by dATP. On the other hand, dotted lines 412, 422, 432, 442, and 452 indicate only the background luminescence caused by dATP because no variation was observed in the luminescence intensity profile after the second to fourth dATP injections.

As shown in FIG. 4A, when the amount of the template DNA is 0.25 pmol and the ratio of dATP to the template DNA is as much as 100 times, almost no variation is observed between the line 411, which indicates luminescence caused by dATP alone, and the line 412, which indicates both of luminescence caused by dATP and luminescence involved in complementary strand synthesis, with an exception of a negligible difference in pattern. It is because the signal intensity involved in complementary strand synthesis is too weak compared with the luminescence intensity caused by dATP. The result of such an experiment that the amount of the template DNA was increased step by step showed that a difference in pattern had gradually appeared between the presence and absence of un-reacted template DNA, luminescence involved in complementary strand synthesis being identified. When the amount of the template DNA was increased to 10 pmol, the ratio of dATP to the template DNA being 2.5 times (f), three types of luminescence intensity profiles were observed unlike other cases. In other words, luminescence after the first dATP injection is indicated by the solid line 461, luminescence after the second dATP injection is indicated by a dashed line 463, and luminescence after the third and fourth dATP injections is indicated by a dotted line 462 because no variation was observed in their luminescence intensity profiles. This suggests that some bases remained un-reacted in the template DNA after the first dATP injection and therefore, the luminescence reaction involved in complementary strand synthesis was observed even after the second dATP injection, though no luminescence involved in complementary strand synthesis was observed after the third and fourth dATP injections, background luminescence alone being reflected in the luminescence intensity profiles.

As shown in the figure, in the luminescence intensity pattern containing the signal intensity involved in complementary strand synthesis, as the amount of the template DNA is increased, the signal intensity involved in complementary strand synthesis becomes gradually noticeable and when the amount of the template DNA is 0.5 pmol or more, namely when the ratio of dATP to the template DNA is 50 times or less, luminescence involved in complementary strand synthesis may be discriminated from background luminescence caused by dATP. It is preferable that the amount of the template DNA is 1.25 pmol or more, at which the signal intensity caused by dATP and the luminescence intensity involved in DNA complementary strand synthesis is at almost the same level, namely the ratio of dATP to the template DNA is 20 times or less. It is further preferable that the ratio is 2.5 to 20 times because complementary strand synthesis comes to completion after one injection when the ratio being 2.5 times or more. Thus, it is clarified that accurate sequencing may be achieved by determining the allowed range of the ratio of the amount of injected dATP to the template DNA.

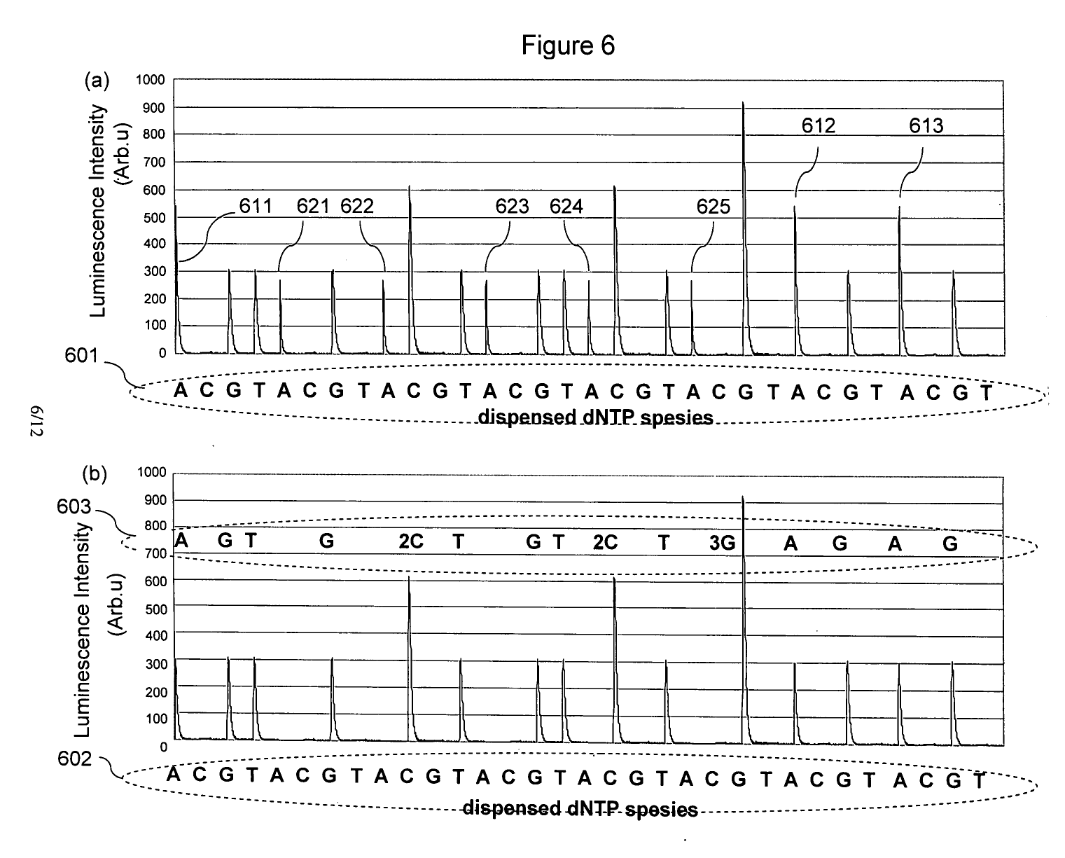

FIG. 6A shows the result of sequencing with dATP. The p53 variant was used for the template DNA. 1 pmol of the p53 variant was used for the template DNA. The amount of dATP was 10 pmol, being equivalent to 10 times the amount of the template DNA. The amount of other dNTPs was 25 pmol. Four kinds of dNTPs were dispensed into the reaction solution containing the mixture of a luminescence reagent, the template DNA, and polymerase in the order of A, C, G, and T. The species of the dispensed bases were listed in the bottom line 601 of FIG. 6A. The p53 variant sequence, AGTGCCT, was determined based on the signal intensity and recorded in the bottom line 602 of FIG. 6B. It should be noted that any repeat sequence was recorded with the number of repeats as “2C”.

The peak intensities were high for a first signal intensity 611, as well as signal intensities 612 and 613 because luminescence involved in DNA complementary strand synthesis and background luminescence caused by dATP were overlapped. On the other hand, signal intensities 621, 622, 623, 624, and 625 reflected background luminescence caused by dATP alone, their peak intensities being low. The background luminescence intensity profile caused by dATP was subtracted from each of the luminescence intensity profiles, which was initiated by injecting dATP, at each of measurement points after dATP injection for correction to obtain the luminescence intensity profile involved in complementary strand synthesis and the obtained luminescence intensity profile was indicated in FIG. 6B. In this way, accurate sequencing may be achieved by removing the effects of dATP luminescence.

As mentioned above, even though dATP which reacts with luciferase and develops the luminescence reaction is used, accurate sequencing may be achieved by optimizing the range of dATP concentrations and subtracting the signal intensity caused by dATP for correction.

The method for determining background luminescence caused by dATP may include three techniques described below. The first one involves a step of injecting dATP into the luminescence reagent with the same condition as in sequence analysis with an exception that no template DNA is mixed, assuming the resulting luminescence intensity profile to be background luminescence caused by dATP.

In the second one, complementary strand synthesis at an site A of the template DNA proceeds sufficiently after dATP was injected several times and no variation is observed in the profiles of luminescence caused by dATP and several profiles of luminescence. The luminescence intensity profile, in which no variation is observed, is assumed to be background luminescence caused by dATP.

In some cases, the number of injections has been determined prior to the initiation of sequencing. In such a case, if A repeats too many times, some template DNA may remain un-reacted. In the third one, background luminescence caused by dATP is estimated by observing background luminescence caused by dATP other than the target background luminescence, in particular those directly before and after the target background luminescence.

Hereinafter, the sequence reaction is described in detail. 5 μL of DNA sample (1 pmol/μL) and 5 μL and an amount, which is 1.5 times that of the DNA sample, of primer were hybridized an annealing buffer (10 mM Tris-acetate buffer, pH7.75, 2 mM magnesium acetate)(94° C., 20s→68° C., 120s→4° C.) to obtain 10 μL (0.5 pmol/μL) of DNA template sample solution. The p53 variant was hybridized under the conditions of 94° C., 20s→65° C., and 120s→4° C. k-ras-Val 1 and the p53 variant were used for the template DNA and these were used for the primers.
k-ras-Val 1 primer: 5′-aag gcact cttgc ctacg cca-3′(SEQUENCE NO.1)
Template DNA: 5′-gactgaat ataaacttgt ggtagttgga gctgttggcg taggcaagag tgccttgacgatacagctaa ttc-3′ (SEQUENCE NO.2)

The analysis clarified a sequence, ac agctc caact accac aagtt tatat tcagt c (SEQUENCE NO.3).
p53 variant primer: 5′-ga acagc tttga ggtgc gtgtt-3′(SEQUENCE NO.4)
Template DNA: 5′-cttc ttgcg gagat tctct tcctc tgtgc gccgg tctct cccag gacag gcact aacac gcacc tcaaa gctgt tccgt cccag tagat tacca accat tagat gaccc tgcct tgtcg aaact ccacg cacaa tcacg gacag gaccc tctct ggccg cgtgt ctcct tctct tagag gcgtt ctttc-3′(SEQUENCE NO.5)

The abovementioned analysis clarified a sequence, agtgc ctgtc ctggg agaga ccggc gcaca gagga agaga atctc cgcaa gaaag (SEQUENCE NO.6).

In sequencing, such a reaction vessel that is composed of reaction cells with 6 mm in inner diameter and 11 mm in depth, being capable of containing up to 300 μL of reaction solution in total was used. The luminescence reagent (20 μL) and the template DNA (2 μL, 1 pmol) were dispensed into the reaction vessel.

The standard composition of the luminescence reagent is shown in Table 1.

TABLE 1
Composition of Luminescence Reagent

0.1 M Tris-acetate (pH 7.7)
2 mM EDTA
10 mM magnesium acetate
0.1% BSA
1 mM dithiothreitol (DTT)
3 μM APS
0.4 mg/mL PVP
0.4 mM D-luciferin
200 mU/mL ATP sulfurylase
2.0 mg/mL luciferase
2 U apyrase VII
0.1 U/μL DNA polymerase I Klenow fragment (exo⁻)

Klenow fragments (Exo-Klenow) with no Exo activity were used for a polymerase enzyme. Klenow fragments with Exo activity may induce a step of cutting off a terminal base in complementary strand synthesis to recombine a complementary strand base. Accordingly, in sequencing using step by step complementary strand synthesis, the identical sequence may be repeatedly read or the complementary strand synthesis reaction may proceed under the different condition for each of DNA copies.

An apparatus used for sequencing is a rotary DNA sequence analysis device, in which four kinds of substrates are sequentially injected into the reaction vessels, originally developed by the inventor. The four reaction vessels are arranged and held by holders on a circumference. The reagent is dispensed by dispensers and the amount of reagent is controlled by means of applied pressure and time period of pressure application. Commercially available chips may be used for the reaction vessels.

50 to 200 μL of any of four kinds of substrate solutions were added in each of the four rotary dispensers and 0.25 μL of dATP with 40 μM of concentration and 0.25 μL of other dNTPs with 100 μM concentration, each were sequentially dispensed into the reaction vessels. To stir the solution in the reaction vessels, a microvibrator was brought in contact with them. Stirring was made for 20 seconds after dNTP injection and then sequencing was carried out at room temperature, 25° C. Light emitted when dNTP was injected into the reaction solution was detected by a photodiode. A detection circuit was high-resistant current/voltage circuit, which was composed of a preamplifier for conversion and a buffer amplifier with 20 times of amplification power. The obtained analog signal intensities were A/D converted and then entered into a computer, based on which the reactions were controlled.

### Example 2

As shown in FIG. 4, almost no difference is observed in the time to reach the peak after dATP injection even though the ratio of dATP to the template DNA. In this case, the luminescence intensity profiles may be compared each other simply by comparing the peak intensities of them. In other words, the luminescence intensity involved in template DNA complementary strand synthesis may be obtained by subtracting luminescence caused by dATP from measured luminescence with the template DNA.

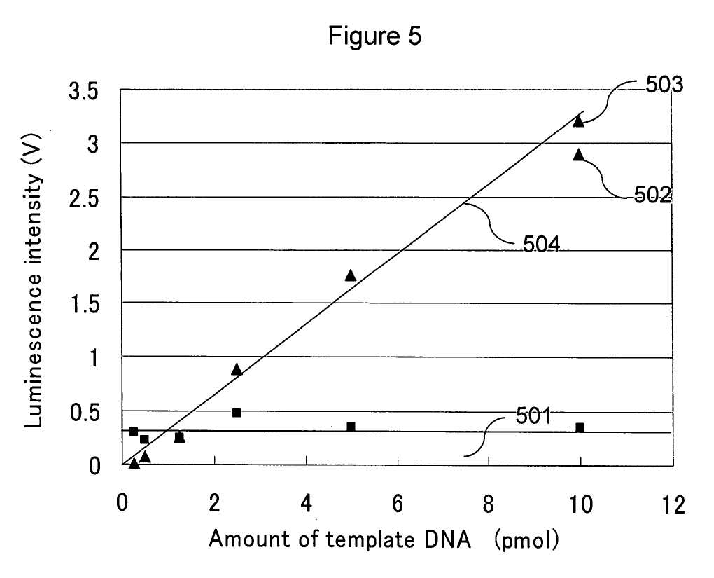

FIG. 5 shows a change in the first peak value after the first dATP injection and changes in the peak values when no variance was observed in the luminescence intensity profiles even though dATP was added as the amount of the template DNA was varied. The peak value when no variation was observed in the luminescence intensity profile even though dATP was added may be approximately represented by means of a line 501. This line suggests that the peak value is almost constant regardless of the amount of the template DNA, reflecting background luminescence caused by dATP only.

The first peak value after dATP injection becomes higher as the amount of the template DNA increases. To extract the luminescence intensity involved in DNA complementary strand synthesis, the peak value of luminescence caused by dATP may be subtracted from the peak value of measured luminescence. The luminescence intensity involved in DNA complementary strand synthesis is on the line 502 with 10 pmol of template DNA, deviating from the line 504 passing though an origin, while being almost along the line 504 with any other amount. With 10 pmol of template DNA, a difference in peak value between the second injection and the third and subsequent injections was considered to reflect luminescence involved in complementary DNA, which did not react with dATP after the first dATP injection, and thereby, this difference was added to 502 to obtain 503, which was assumed to be the total amount of luminescence intensities involved in DNA complementary strand synthesis. Thus, it is clarified that all the peak values of luminescence involved in complementary strand synthesis are almost along the line 504.

The base sequences may be obtained by subtracting the peak value of measured luminescence caused by dATP and the peak value of background luminescence. The result is shown in Table 2.

TABLE 2
Measured Peak Value of Luminescence Intensity caused by
dATP and Corrected Value Subtracting the Background

| | Peak position in FIG. 6 | | | | | | | |
|---|---|---|---|---|---|---|---|---|
| | 611 | 621 | 622 | 623 | 624 | 625 | 612 | 613 |
| Measured | 540 | 272 | 267 | 271 | 268 | 270 | 538 | 541 |
| peak value | | | | | | | | |
| (Arb. u) | | | | | | | | |
| Corrected | 270 | 2 | −3 | 1 | −2 | 0 | 268 | 271 |
| value | | | | | | | | |
| (Arb. u) | | | | | | | | |

### Example 3

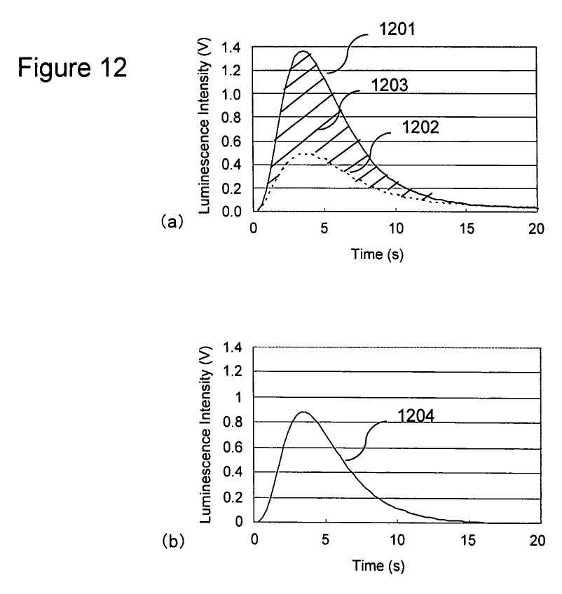

The measured luminescence intensity profiles is indicated by a solid line 1201 and the background luminescence intensity profile by a dotted line 1202, a region between lines 1201 and 1202 being filled by slant lines 1203 in FIG. 12A. The region 1203 filled with slant lines indicates a difference between the measured luminescence intensity profiles and the background luminescence intensity profile. The measured luminescence intensity profile and the background luminescence intensity profile were subtracted at each measurement point for correction to obtain the luminescence intensity profile involved in complementary strand synthesis and the obtained profile was indicated by means of a solid line 1204 in FIG. 12B. The data shown in FIGS. 6A and 6B were also obtained in the same way.

Alternatively, the base sequences may be determined by obtaining the area of the peak region for each of injected base species and comparing the obtained areas.

### Example 4

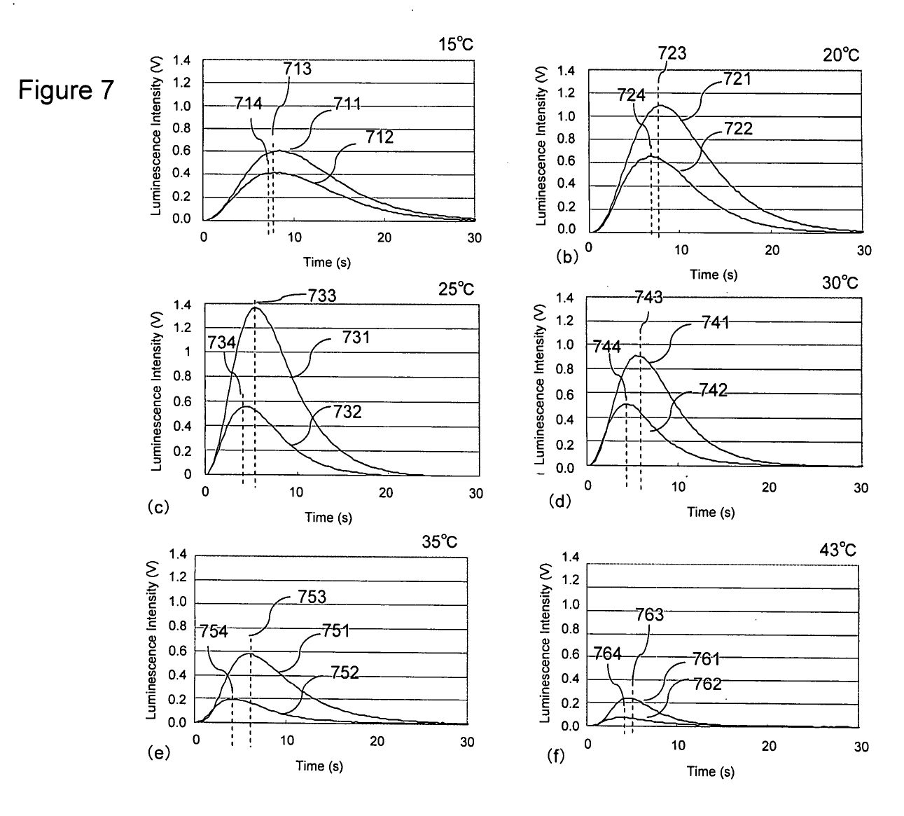

As shown in FIGS. 1 and 2, the pyrosequenceing used in this example involves four enzymatic reaction systems. The luminescence intensity profile involved in the luciferase reaction had differences in peak intensity, peak width, and peak shape because four enzymatic reaction systems competed with each other, with peak points shifted each other in time. The activity of enzymatic reaction depends on its environmental conditions such as temperature and pH. Herein, variations in luminescence intensity profile with the temperature varied as an example of environmental conditions is shown in FIGS. 7A to 7F. It was assumed that the amount of dATP was 25 pmol and 2.5 pmol of p53 variant was used for the template DNA. The amount of dATP is equivalent to ten times that of the template DNA. In FIG. 7A, the result of the experiment at 15° C. is shown and the luminescence intensity and the position of the peak intensity with the template DNA were indicated by 711 and 713, respectively, while the luminescence intensity and the position of the peak intensity without the template DNA were indicated by 712 and 714, respectively. Similarly, the profiles of luminescence intensities with or without the template DNA at 20° C., 25° C., 30° C., 35° C., and 43° C. are indicated in FIGS. 7A, 7B, 7C, 7D, 7E, and 7F, respectively.

As known from the aforementioned results, the time to reach the peak after dATP injection depended on the presence or absence of the template DNA and the ambient temperature.

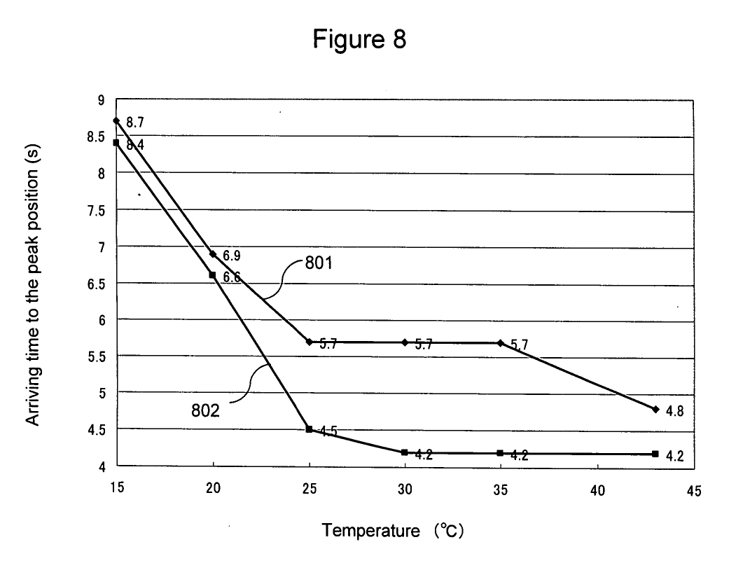

A variation in time to reach the peak after dATP injection is also indicated on a graph in FIG. 8. The time to reach the peak is numerically indicated at each of points in the figure. The arriving times to the peak dependent on the temperature with or without the template DNA is indicated by lines 801 and 802, respectively. As shown in the figure, the arriving time to the peak after dATP injection becomes shorter as the temperature rises regardless of the presence of the template DNA. The comparison of the arriving time to the peak between two conditions, the presence and absence of the template DNA at the same temperatures showed that it is shorter without the template DNA than with the template DNA.

The difference in peak intensity is not so large between two conditions, with the template DNA and without the template DNA at low temperatures such as 15° C. and 20° C., though it becomes larger as the temperature gradually rises to 25° C., 30° C., and 35° C. At a high temperature 43° C., the difference between them becomes smaller. It suggests that four kinds of enzymes have different dependency of reaction rate on temperature.

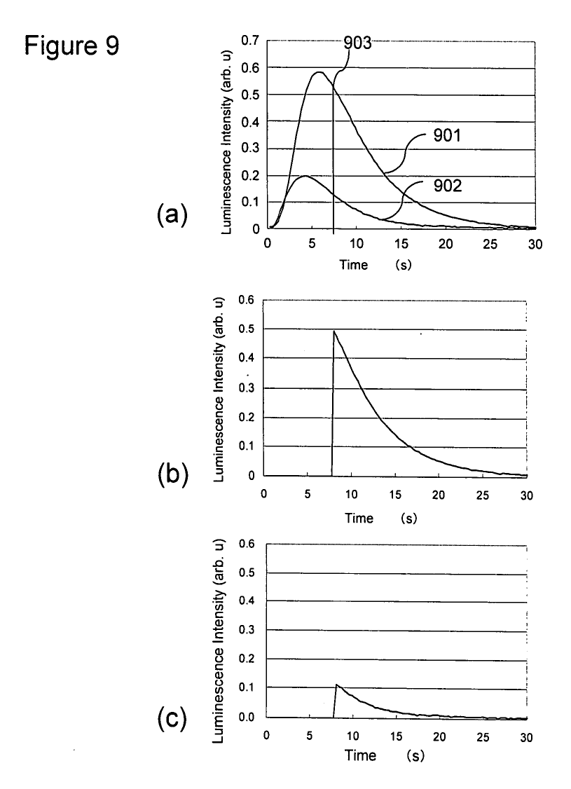

By reference to FIG. 8, the difference in the arriving time to the peak between two conditions, the presence of the template DNA and the absence of the template DNA becomes the largest value 1.5 sec. at arrange of temperatures 30° C. to 35° C. A relatively large difference is also observed at 25° C. and 43° C. The difference is used to discriminate background luminescence intensity profile caused by dATP from the measured luminescence intensity profile. Using a simple method in which the peak background luminescence intensity is assumed to be 0 (zero), any effect of background luminescence may be minimized. In other words, the measured luminescence intensity profile is compared with the background luminescence intensity profile caused by dATP to detect the point where the peaks in both the profiles may be suitably discriminated. The arriving time to the point after dATP injection is assumed to be the cut off time and the luminescence intensity profile up to the cut off time to be 0 (zero), and the profiles of luminescence observed since the cut off time to be the signal intensity of luminescence involved in complementary strand synthesis. Hereinafter, the principle is described by reference to FIG. 9.

The profile 901 of measured luminescence intensity and the profile 902 of background luminescence intensity caused by dATP are shown in FIG. 9A. Eight seconds, after which in the profile 902 of luminescence intensity caused by dATP, the intensity attenuated to ½ times the peak value, were assumed to be the cut off time and indicated by means of 903. In the luminescence intensity profile with the template DNA, the luminescence intensity profile before the cut off time was assumed to be 0 (zero) and indicated in FIG. 9B. In this case, the peak intensity attenuated only by 15% and therefore, it may be assumed to be luminescence involved in complementary strand synthesis. In FIG. 9C, the luminescence intensity profiles before the cut off time among the luminescence intensity profiles caused by dATP were assumed to be 0 (zero). In this example, the attenuation of peak intensity was assumed to be 1/2 the peak of background luminescence intensity caused by dATP but other suitable values may be used.

Thus, by removing the signal intensity up to the cut off time from DATP injection, namely within eight seconds after dATP injection in this example, the signal intensity involved in complementary strand synthesis may be obtained. Using such an advantage in that background luminescence caused by dATP shifts in time from luminescence involved in DNA complementary strand synthesis to suppress background luminescence caused by dATP, high-sensitivity DNA sequencing may be achieved.

The cut off time depends on the conditions such as temperature, pH, and the kind of the enzyme. Thus, the cut off time must be determined depending on the experimental conditions and the experiment is conducted using such cut off time.

In this example, a method using a shift in time due to the temperature dependency of the enzyme activity is described. However, the enzyme activity may change depending on other conditions including pH and the composition of the reagent to be added. For example, conventionally, APS has been used as a substrate for producing ATP from PPi. Alternatively, any other reaction system in which the enzyme activity may change depending on the conditions may be used to produce ATP from PPi. For example, AMP (Adenosine monophosphate) may be used as the substrate to produce ATP from PPi and PPDK (Pyruvate orthophosphate dikinese) may be used for the enzyme.

### Example 5

In this example, luminescence intensity involved in complementary strand synthesis was obtained by reducing the amount of dATP to some degree and subtracting background luminescence intensity caused by dATP from measured luminescence intensity. This example relates to the method for obtaining the luminescence intensity involved in complementary strand synthesis by repeating a step of injecting dATP into the reaction vessel several times and using a variation in the obtained chemical luminescence intensity.

This example was carried out at room temperature of 25° C. to obtain higher peak intensity. For example, when an amount of dATP that is five times the amount of the template DNA is injected in the step by step sequencing, the luminescence intensity of the DNA sequence including repeats of A (adenine) is higher than that of the DNA sequence including single A. Usually, the DNA sequence includes less than three repeats of A. In such a case complementary strand synthesis can be almost completed after the first injection of dATP, if the amount of dATP that is five times amount of the template DNA is injected. In case that the DNA sequence includes more than three repeats of A, un-reacted sites may be left in the template DNA due to the lack of substrate. To solve this problem, in this example, the complete sequencing of the DNA including repeats of A was achieved by several injections of dATP.

In addition to the complete sequencing of the repeat bases by several injections of dATP, this method has an advantage in that the presence or absence of un-reacted sites may be checked. In other words, if dATP is injected several times when the DNA sequence includes three repeat bases, the luminescence intensity after the second injection is at the same level as that of background luminescence intensity caused by dATP. In this way, both the background luminescence intensity and the absence of un-reacted site in the template DNA may be checked.

As described above, the complete sequencing of DNA including up to three repeats of A can be achieved by single injection. However, this is just an example, the result may depend on the amount of dATP and other conditions. It was demonstrated that the rate, at which complementary strand synthesis proceeded, depended largely on the rate of dATP to the template DNA with no significant deviation from the standard conditions and not so greatly on other conditions such as apyrase concentration, temperature, and luciferase concentration.

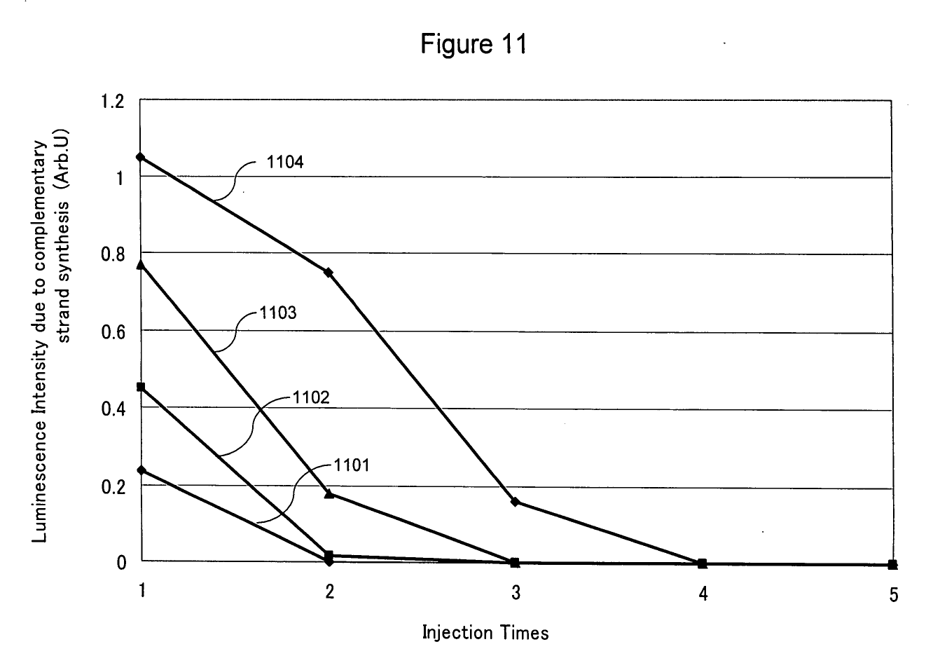

FIG. 11 shows a graph of the luminescence intensity involved in complementary strand synthesis depending on the number of injection when the number of A repeats in the template DNA was varied. The template DNA in 1101 include A at the terminal site, the numbers of repeat base in the template DNA are two (AA) in 1102, four (AAAA) in 1103, and eight in 1104. The number of injections was up to five. Background luminescence intensity caused by dATP was previously subtracted. The amount of template DNA was 5 pmol and the amount of each dATP injection was 25 pmol, which was equivalent to the five times that of the template DNA. When the number of repeat base was one, no luminescence intensity involved in complementary strand synthesis was observed after the second dATP injection, suggesting that the complementary strand synthesis completed after the first dATP injection. A slight amount of luminescence involved in complementary strand synthesis was observed when the number of repeat was two, luminescence involved in complementary strand synthesis of approximately ¼ un-reacted DNA was observed when the number of repeats was four, and the complementary strand synthesis completed after the third dATP injection. When the number of repeats was eight, luminescence intensity involved in complementary strand synthesis of further more un-reacted DNA was observed after up to the third dATP injection.

The integrated values of peak luminescence intensity involved in complementary strand synthesis were indicated in Table 3. The luminescence intensity involved in complementary strand synthesis of one base was 0.238(Arb.U) and therefore, based on this value, the peak luminescence intensities were 1.953, 3.98, and 8.21. Thus, the numbers of repeat bases were determined as two, four, and eight, respectively.

TABLE 3
Estimation of the Number of Repeat Bases

| | Total | Relative | Base |
|---|---|---|---|
| | (Arb. U) | value | number |
| A1 | 0.238 | 1 | 1 |
| A2 | 0.465 | 1.953 | 2 |
| A4 | 0.949 | 3.98 | 4 |
| A8 | 1.959 | 8.21 | 8 |

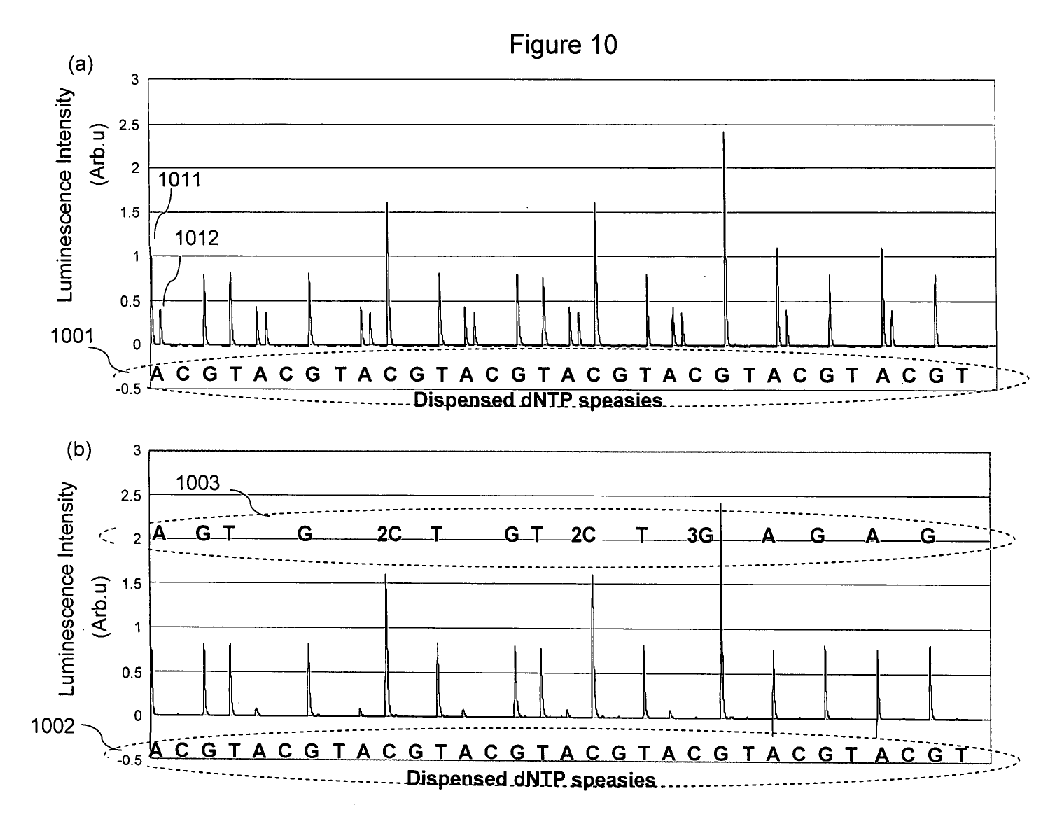

In the following experiment, the sequencing was carried out by injecting an amount of dATP that is five times the amount of the template DNA to the reaction vessel several times (usually, two or three times). The amount of DNA was 0.5 pmol. 0.25 μL of dATP was injected two times consecutively in the reaction vessel for each of bases. The concentration of dATP was 10 μM (the amount for each injection was 2.5 pmol equivalent to 5 times that of the template DNA) and the concentration of substrates other than dATP (e.g. dTTP) was 20 μM (5 pmol equivalent to 10 times that of the template DNA). dATP and other substrates were injected into the reaction vessels two times consecutively to carry out sequencing. The result of sequencing is shown in FIG. 10A.

If the complementary strand synthesis reaction sufficiently proceeds by the first injection of the reaction reagents (1011), the signal intensity generated by the first injection reflects the total of the chemical luminescence intensity caused by ATP generated from PPi (luminescence intensity involved in complementary strand synthesis) and the chemical luminescence intensity caused by the injected reagents (background luminescence intensity caused by dATP). As the complementary strand synthesis was completed by the first injection, the background luminescence after the second injection of the same base (1012) is considered that caused by dATP, which was produced through the luminescence reaction induced by the injected reagents. Accordingly, the difference between two luminescence intensities may be relevant to the luminescence intensity involved in DNA complement strand synthesis. The luminescence intensity corrected based on the above result is shown in FIG. 10B.

In a luminescence intensity pattern (pyrogram) shown in FIG. 10A, any variation in signal intensity caused through a step of subtracting the signal intensity after the second injection severely affects the result. Any variation in luminescence reaction observed after dATP injection depends strongly on a variation in injection amount. Accordingly, it is required that the variation in the amount of injected reagent should be sufficiently reduced compared with that in the signal intensity involved in complementary strand synthesis. On the other hand, the variation in amount of injected reagent is 5% or less of the amount of injected reagent. Therefore, when the amount of dATP is 10 times that of the template DNA or less, the variation causes no problem at all.

If complementary strand synthesis does not sufficiently proceed by the first reagent injection (for example, in case that DNA tends to take a three-dimensional structure or a large amount of dATP is incorporated in the complementary strand synthesis at a time), it is necessary to increase the amount of injected dATP. This may be achieved by increasing the amount for each injection or the number of injections. When the amount for each injection is increased, the signal intensity of the background luminescence becomes too strong, deteriorating the precision, at which the signal intensity involved in complementary strand synthesis is obtained by subtracting the signal intensity of the background luminescence. To solve this problem, the number of injections was increased in this example. By decreasing the amount for each injection and increasing the number of injections, the complementary strand synthesis may be facilitated without increasing the background signal intensity. Thus, the signal intensity involved in complementary strand synthesis may be discriminated from the signal intensity of the background luminescence.

In this example, the apyrase degradation reaction was used to remove dNTP used in the reaction. However, dNTP may be removed by: immobilizing the template DNA and enzymes on the surfaces of solid support (e.g. beads or the like), discarding the reaction solution containing dNTP, washing the reaction vessel, and injecting the flesh solution including reaction reagent such as dNTP. This method has an advantage in that more accurate and stable sequencing can be achieved by avoiding the accumulation of reaction products or the like although it may be complicated.

What is claimed is:

1. A method for sequencing a template nucleic acid, comprising the steps of:
adding an amount of dATP that dose not exceed 50 times the amount of a template DNA to a reaction vessel containing the template DNA for step by step complementary strand synthesis; and
subtracting the background luminescence intensity caused by dATP, which is a substrate for luciferase, from the measured luminescence intensity, and
measuring the luminescence intensity involved in the complementary strand synthesis, wherein said luminescence intensity is caused by ATP, which is produced from pyrophosphoric acid generated through the complementary strand synthesis.

2. The method of claim 1, wherein the amount of dATP to be injected into the reaction vessel does not exceed 20 times the amount of the template DNA.

3. The method of claim 1, wherein the luminescence intensity involved in the complementary strand synthesis is measured by detecting a point at which a measured luminescence intensity profile can be suitably discriminated from a background luminescence intensity profile caused by dATP, and removing the luminescence intensity profile up to that point to obtain the luminescence intensity involved in the complementary strand synthesis.

4. The method of claim 1, wherein the luminescence involved in the complementary strand synthesis is measured by: removing any luminescence intensity within a cut off time period to obtain the luminescence intensity involved in the complementary strand synthesis, wherein the cut off time period indicating the time period required to reach a point at which the luminescence intensity profile can be suitably discriminated from the background luminescence intensity profile.

5. The method of claim 4, wherein the cut off time period is eight seconds.

6. The method of claim 4, wherein the complementary strand synthesis is performed at 30° C. to 43° C.

7. The method of claim 1, wherein the luminescence intensity involved in the complementary strand synthesis is measured by injecting dATP into the reaction vessel at least two times, confirming the background luminescence intensity caused by dATP every time dATP is injected, and subtracting the background luminescence intensity caused by dATP from the measured luminescence intensity.

8. The method of claim 1, wherein excessive amounts of dNTP are removed after the complementary strand synthesis.

9. The method of claim 8, wherein the nucleic acid substrates are removed with the aid of an enzyme mixed therewith in the reaction vessel.

10. The method of claim 8, wherein the nucleic acid substrates are removed by replacing a reaction solution with flesh solution after immobilizing a template DNA and a nucleic acid synthase.
